(19) United States
(12) Patent Application Publication
(10) Pub. No.: US 2004/0235088 A1
(43) Pub. Date: Nov. 25, 2004

(54) Process for the production of ergosterol and its intermediate products using recombinant yeasts

(75) Inventors: Alfred Weber, Berlin (DE); Uwe Klages, Berlin (DE); Mario Kennecke, Berlin (DE); Christine Lang, Berlin (DE); Ulf Stahl, Berlin (DE); Thomas Polakowski, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering Aktiengesellschaft, Berlin (DE)

(21) Appl. No.: 10/665,449

(22) Filed: Sep. 22, 2003

Related U.S. Application Data
(63) Continuation of application No. 09/509,608, filed on Jun. 19, 2000, now abandoned.
Application No. 09/509,608 is a 371 of international application No. PCT/EP98/06134, filed on Sep. 28, 1998.

(30) Foreign Application Priority Data
Sep. 30, 1997 (DE) 197 44 212.9

(51) Int. Cl. C12P 33/00; C12N 1/18; C12N 15/74

(52) U.S. Cl. 435/52; 435/483; 435/254.2

(57) ABSTRACT

A process for the production of ergosterol and its intermediate products using recombinant yeasts and plasmids for transformation of yeasts is described.

This invention relates to a process for the production of ergosterol and its intermediate products using recombinant yeasts and plasmids for the transformation of yeasts.

Ergosterol is the end product of sterol synthesis in yeasts and fungi. The economic importance of this compound lies, on the one hand, in obtaining vitamin D₂ from ergosterol with UV irradiation, and, on the other hand, in obtaining steroid hormones with biotransformation, starting from ergosterol. Squalene is used as a synthesis component for the synthesis of terpenes. In hydrogenated form, it is used as squalene in dermatology and cosmetics and in various derivatives as components of skin and hair cleansers. Also of economic importance are the intermediate products of the ergosterol metabolic process. Farnesol, geraniol and squalene can be named as most important here. In addition, sterols, such as, e.g., zymosterol and lanosterol, can be used economically, whereby lanosterol is pivotal in terms of crude and synthesis for the chemical synthesis of saponins and steroid hormones. Because of its good skin penetration and spreading properties, lanosterol is used as an emulsion adjuvant and active ingredient for skin creams.

The genes of the ergosterol metabolism in yeast are largely known and cloned, e.g., the HMG-COA reductase (HMG1) (Basson et al. (1988)), the squalene synthetase (ERG9) (Fegueur et al. (1991)), the acyl-CoA: sterol-acyl transferase (SAT1) (Yu et al. (1996)), and the squalene epoxidase (ERG1) (Jandrositz et al. (1991)). Squalene synthetase catalyzes the reaction of farnesyl pyrophosphate on presqualene pyrophosphate to squalene. The reaction mechanisms of sterol-acyl transferase are not fully determined. An over-expression of genes of these above-mentioned enzymes was already attempted, but it did not result in any significant increase in the amount of ergosterol. In the case of the HMG1 over-expression, the overproduction of squalene was described; moreover, additional mutations were introduced to interrupt the route following squalene (EP-0 486 290).

The overproduction of geraniol and farnesol was also described, but here no over-expression of genes of the ergosterol metabolism took place, rather an interruption of the reaction process as regards geraniol and farnesol formation (EP-0313 465).

Specific inhibitors of the ergosterol biosynthesis can also result in the accumulation of larger amounts of certain intermediate products, e.g., allylamines, which prevent the conversion of squalene into squalene epoxide. As a result, large amounts (up to 600 times the normal level) of squalene are accumulated (Jandrositz et al., (1991)).

Although the use of inhibitors leads to a major accumulation of, e.g., squalene, the addition of these substances may yet turn out to be disadvantageous since only small amounts exert the same action in the organism, so that a production of the products of ergosterol biosynthesis in the process of overproduction is advantageous.

The object of this invention is to synthesize a microbiological process for the production of ergosterol and its intermediate products, the microorganisms that are necessary for this purpose, such as yeast strains, the increased amounts of ergosterol or intermediate products that are necessary for this purpose, and to prepare the plasmids that are necessary for the transformation of the yeast strain.

It was now found that the amount of ergosterol and its intermediate products can be increased, if the genes of HMG1 (Basson et al., (1988)), ERG9 (Fegueur et al., (1991)), Current Genetics 20: 365-372), SAT1 (Yu et al., (1996)) and ERG1 (Jandrositz et al. (1991)) are introduced in altered form into microorganisms such as, e.g., yeasts, whereby the genes are located either individually on a plasmid or in combination on one or more plasmids and can be brought to the host simultaneously or in succession.

The subject of this invention is thus a process that is characterized in that

a) first a plasmid is designed, into which several suitable genes of the ergosterol metabolic process are inserted in altered form, or

b) first plasmids are designed, into which in each case one of the genes of the ergosterol metabolic process is inserted in altered form,

c) microorganisms are transformed with the thus produced plasmids, whereby the microorganisms are transformed with a plasmid under a) or they are transformed simultaneously or in succession with several plasmids under b),

d) fermentation into ergosterol is performed with the thus produced microorganisms,

e) after fermentation has ended, the ergosterol and its intermediate products are extracted from the cells and analyzed, and finally,

f) the thus obtained ergosterol and its intermediate products are purified using column chromatography and isolated.

The subject of this invention is especially a process which is characterized in that

a-i) first a plasmid is designed, into which the following genes are inserted:

i) the gene of HMG-Co-A-reductase (t-HMG),

ii) the gene of squalene synthetase (ERG9),

iii) the gene of Acyl-CoA: sterol-acyl transferase (SAT1), and

iv) the gene of squalene epoxidase (ERG1), or

a-ii) first a plasmid is designed, into which the following genes are inserted:

i) the gene of HMG-Co-A-reductase (t-HMG), and

ii) the gene of squalene synthetase (ERG9), or

a-iii) first a plasmid is designed, into which the following genes are inserted:

i) the gene of HMG-Co-A-reductase (t-HMG), and

iii) the gene of acyl-CoA: sterol-acyl transferase (SAT1), or

a-iv) first a plasmid is designed, into which the following genes are inserted:

i) the gene of the HMG-Co-A-reductase (t-HMG), and

iv) the gene of squalene epoxidase (ERG1), or

a-v) first a plasmid is designed, into which the following genes are inserted:

ii) the gene of squalene synthetase (ERG9), and

iii) the gene of acyl-CoA: sterol-acyl transferase (SAT1) or

a-vi) first a plasmid is designed, into which the following genes are inserted:

ii) the gene of squalene synthetase (ERG9), and

iv) the gene of squalene epoxidase (ERG1), or

a-vii) first a plasmid is designed, into which the following genes are inserted:

iii) the gene of acyl-CoA: sterol-acyl transferase (SAT1), and

iv) the gene of squalene epoxidase (ERG1), or

b) first plasmids are designed, into which in each case one of the genes that is mentioned under a-i) is inserted, and

c) microorganisms are transformed with the thus produced plasmids, whereby the microorganisms are transformed with a plasmid under a-i) to a-vii), or they are transformed simultaneously or in succession with several plasmids under b),

d) fermentation into ergosterol is performed with the thus produced microorganisms,

e) after fermentation has ended, the ergosterol and its intermediate products are extracted from the cells and analyzed, and finally

f) the thus obtained ergosterol and its intermediate products are purified using column chromatography and isolated.

In addition, the gene of squalene epoxidase (ERG1) can be inserted into the plasmids that are cited under a-ii), a-iii) and a-v), and in addition, the gene of acyl-CoA: sterol-acyl transferase (SAT1) can be inserted into the plasmid that is cited under a-ii). These plasmids are also subjects of this invention.

Intermediate products are defined as squalene, farnesol, geraniol, lanosterol, zymosterol, 4,4-dimethylzymosterol, 4-methylzymosterol, ergost-7-enol and ergosta-5,7-dienol, especially sterols with 5,7-diene structure.

The plasmids that are used are preferably the plasmid YEpH2, which contains the average ADH-promoter, t-HMG (altered variant of HMG1) and the TRP-terminator (see FIG. 1), the plasmid YDpUHK3, which contains the average ADH-promoter, t-HMG (altered variant of HMG1) and the TRP-terminator, the gene for the kanamycin resistance and the ura3 gene (see FIG. 2) and the plasmid pADL-SAT1, which contains the SAT1 gene and the LEU2 gene of YEp13.

These plasmids and their use for the production of ergosterol and its intermediate products, such as squalene, farnesol, geraniol, lanosterol, zymosterol, 4,4-dimethylzymosterol, 4-methylzymosterol, ergost-7-enol and ergosta-5,7-dienol, especially sterols with 5,7-diene structure, are also subjects of this invention.

As a host for the introduction of plasmids according to the invention, in principle all microorganisms, especially yeasts, are suitable.

The species S. cerevisiae, especially the strain S. cerevisiae AH22, is preferred.

The subject of this invention is also the yeast strain S. cerevisiae AH22, which contains one or more of the genes that are mentioned in the process under a-i).

The subject of this invention is also the yeast strain S. cerevisiae AH22, which contains the plasmid pADL-SAT1.

In addition, the combined transformation of microorganisms with the plasmids pADL-SAT1 and YDpUHK3, especially yeasts such as S. cerevisiae AH22, is preferred.

Viewed overall, the flow in the ergosterol metabolic process is affected as follows:

The flow in the direction of ergosterol is maximized by the activity of several bottle-neck enzymes being intensified simultaneously. In this case, various enzymes play a decisive role, whereby the combination of deregulation or over-expression provides the decisive breakthrough for increasing the ergosterol yield. As combinations, the enzymes or their genes HMG1 (Basson et al., (1988)), ERG9 (Fegueur et al., (1991)), acyl-CoA: sterolacyl transferase (SAT1) (Yu et al. (1996)) and/or squalene epoxidase (ERG1) (Jandrositz et al. (1991)) are introduced into a yeast strain in altered form, whereby the genes are introduced with one or more plasmids, whereby the DNA sequences are contained either individually or in combination in the plasmid(s).

In the case of gene HMG1, “altered” means that of the corresponding genes, only the catalytic area is expressed without the membrane-bound domains. This alteration was already described (EP-0486 290). The purpose of the alteration of HMG1 is to prevent the feedback regulation by intermediates of ergosterol biosynthesis. Both HMG1 and the two other above-mentioned genes are removed in the same way from the transcriptional regulation. To this end, the promoter of the genes is replaced by the “average” ADH1-promoter. This promoter fragment of the ADH1-promoter shows an approximately constitutive expression (Ruohonen et al., (1995)), so that the transcriptional regulation no longer proceeds via intermediates of the ergosterol biosynthesis.

The products that are produced in the over-expression can be used in biotransformations or other chemical and therapeutic purposes, e.g., obtaining vitamin D₂ from ergosterol via UV irradiation, and obtaining steroid hormones via biotransformation starting from ergosterol.

Subjects of this invention are also microorganisms, especially yeast strains, which can produce an increased amount of ergosterol and ergosterol in combination with increased amounts of squalene by over-expression of the genes that are mentioned in the process under a-i).

Preferred is an altered variant of the gene HMG1, in which only the catalytic area is expressed without the membrane-bound domain. This alteration is described (EP-0486 290).

A subject of this invention is also a process for the production of ergosterol and its intermediate products, which is characterized in that the genes that are mentioned in the process under a), especially the genes that are mentioned in the processes under a-i to a-vii) (two-, three-, and four-fold gene combinations) in each case with the plasmids are first introduced independently of one another into microorganisms of the same species, and fermentation into ergosterol is performed with them together, and the ergosterol that is thus obtained is extracted from the cells, analyzed and purified using column chromatography and isolated.

Subjects of this invention are also expression cassettes, comprising the average ADH-promoter, the t-HMG gene, the TRP-terminator, and the SAT1-gene with the average ADH-promoter and the TRP-terminator and expression cassettes, comprising the average ADH-promoter, the t-HMG gene, the TRP-terminator, the SAT1 gene with the average ADR-promoter and the TRP-terminator, and the ERG9-gene with the average ADH-promoter and the TRP-terminator.

A subject of this invention is also a combination of expression cassettes, whereby the combination consists of

a) a first expression cassette, in which the ADH-promoter, the t-HMG gene and the TRP-terminator are located,

b) a second expression cassette, in which the ADH-promoter, the SAT-1 gene and the TRP-terminator are located, and

c) a third expression cassette, in which the ADH-promoter and the ERG9-gene with the TRP-terminator are located.

The subject of this invention is also the use of these expression cassettes for the transformation of microorganisms, which are used in the fermentation into ergosterol, whereby the microorganisms are preferably yeasts.

Microorganisms such as yeasts, which contain these expression cassettes, as well as their use in the fermentation into ergosterol and ergosterol intermediate products, are also subjects of the invention.

The following examples are used for the explanation with respect to the implementation of the processes that are necessary for the embodiments:

1. Restriction

The restriction of plasmids (1 to 10 μg) was performed in 30 μl batches. To this end, the DNA was taken up in 24 μl of H₂O, and mixed with 3 μl of the corresponding buffer, 1 μl of RSA (bovine serum albumin) and 2 μl of enzyme. The enzyme concentration was 1 unit/μl or 5 units/μl depending on the amount of DNA. In some cases, 1 μl more of RNase was added to the batch to degrade the tRNA. The restriction batch was incubated for two hours at 37° C. The restriction was controlled with a minigel.

2. Gel Electrophoreses

The gel electrophoreses were performed in minigel or wide-minigel equipment. The minigels (about 20 ml, 8 bags) and the wide-minigels (50 ml, 15 or 30 bags) consisted of 1% agarose in TAE. 1×TAE was used as a mobile buffer. The samples (10 μl) were mixed with 3 μl of stopper solution and applied. I-DNA cut with HindIII was used as a standard (bands at: 23.1 kb; 9.4 kb; 6.6 kb; 4.4 kb; 2.3 kb; 2.0 kb; 0.6 kb). For separation, a voltage of 80 V for 45 to 60 minutes was prepared. Then, the gel was stained in ethidium bromide solution and held under UV light with video-documentation system INTAS or photographed with an orange filter.

3. Gel Elution

The desired fragments were isolated using gel elution. The restriction preparation was applied in several bags of a minigel and separated. Only λ-HindIII and a “sacrifice trace” were stained in ethidium bromide solution, viewed under UV light, and the desired fragment was labeled. As a result, DNA was prevented from damaging the residual bags by the ethidium bromide and the UV light. By aligning the stained and unstained gel pieces, the desired fragment from the unstained gel piece could be cut out based on the labeling. The agarose piece with the fragment to be isolated was added in a dialysis tube, sealed free of air bubbles with a little TAE buffer and placed in the BioRad-minigel apparatus. The mobile buffer consisted of 1×TAE, and the voltage was 100 V for 40 minutes. Then, the flow polarity was varied for 2 minutes to loosen the DNA adhering to the dialysis tube. The buffer that contains the DNA fragments of the dialysis tube was moved into the reaction vessel and thus performed an ethanol precipitation. To this end, 1/10 volume of 3 M sodium acetate, tRNA (1 μl per 50 μl of solution) and 2.5 times the volume of ice-cold 96% ethanol were added to the DNA solution. The batch was incubated for 30 minutes at −20° C. and then centrifuged off at 12,000 rpm for 30 minutes at 4° C. The DNA pellet was dried and taken up in 10 to 50 μl of H₂O (depending on the amount of DNA).

4. Klenow Treatment

Projecting ends of DNA fragments are made up by the Klenow treatment, so that “blunt ends” result. Per 1 μg of DNA, the following batch was pipetted together:

$$DNA \text{-} pellet + 11 μ 1 of {H}_{2} O + 1.5 μ 1 of 10 \times Klenow buffer + 1 μ 1 of 0.1 M DTT + 1 μ 1 of nucleotides ( dNTP 2 mmol ) + 1 μ 1 of Klenow \text{-} polymerase ( 1 unit \text{/} μ 1 )$$

In this case, the DNA should be derived from an ethanol precipitation to prevent contaminants from inhibiting the Klenow-polymerase. Incubation was carried out for 30 minutes at 37° C., and then over another 5 minutes at 70° C. the reaction was halted. The DNA was obtained from the batch by an ethanol precipitation and taken up in 10 μl of H₂O.

5. Ligation

The DNA fragments that were to be ligated were combined. The end volume of 13.1 μl contained about 0.5 μg of DNA with a vector-insert ratio of 1:5. The sample was incubated for 45 seconds at 70° C., cooled to room temperature (about 3 minutes) and then incubated on ice for 10 minutes. Then, the ligation buffers were added: 2.6 μl of 500 mmol TrisHCl, pH 7.5, and 1.3 μl of 100 mmol MgCl₂, and they were incubated on ice for another 10 minutes. After 1 μl of 500 mmol DTT and 1 μl of 10 mmol ATP were added, 1 μl of ligase (1 unit/μl) was added on ice for another 10 minutes. The entire treatment should be carried out with as little shaking as possible so as to keep adjacent DNA ends from reseparating, The ligation was carried out overnight at 14° C.

6. E. coli Transformation

Component Escherichia coli (E. coli) NM522 cells were transformed with the DNA of the ligation preparation. As a positive control, a batch was supplied with 50 ng of the pScL3 plasmid, and as a null control, a batch was supplied without DNA. For each transformation preparation, 100 μl of 8% PEG solution, 10 μl of DNA and 200 μl of competent cells (E. coli NM522) were pipetted into a tabletop centrifuging tube. The batches were put on ice for 30 minutes and shaken intermittently. Then, thermal shock took place: 1 minute at 42° C. For regeneration, 1 ml of LB-medium was added to the cells and incubated on a shaker for 90 minutes at 37° C. 100 μl each of the undiluted batches, a 1:10 dilution and a 1:100 dilution were flattened out on LB+ampicillin plates and incubated overnight at 37° C.

7. Plasmid Isolation from E. Coli (Miniprep)

E. coli colonies were cultured overnight in 1.5 ml of LB+ampicillin medium in tabletop centrifuging tubes at 37° C. and 120 rpm. The next day, the cells were centrifuged off for 5 minutes at 5000 rpm and 4° C., and the pellet was taken up in 50 μl of TE-buffer. Each batch was mixed with 100 μl of 0.2N NaoH, 1% SDS solution, mixed and put on ice for 5 minutes (lysis of the cells). Then, 400 μl of Na-acetate/Nacl solution (230 μl of H₂O, 130 μl of 3 M sodium acetate, and 40 μl of 5 M NaCl) was added, the batch was mixed and put on ice for another 15 minutes (protein precipitation). After 15 minutes of centrifuging at 11,000 rpm, the supernatant, which contains plasmid-DNA, was moved into an Eppendorf vessel. If the supernatant was not completely clear, it was centrifuged one more time. The supernatant was mixed with 360 μl of ice-cooled isopropanol and incubated for 30 minutes at −20° C. (DNA precipitation). The DNA was centrifuged off (15 minutes, 12,000 rpm, 4° C.), the supernatant was discarded, the pellet was washed in 100 μl of ice-cooled 96% ethanol, incubated for 15 minutes at −20° C. and centrifuged off again (15 minutes, 12,000 rpm, 4° C.). The pellet was dried in a speed vacuum and then taken up in 100 μl of H₂O. The plasmid-DNA was characterized by restriction analysis. To this end, 10 μl of each batch was restricted and cleaved by gel electrophoresis in a wide-minigel (see above).

8. Plasmid-working-Up on E. coli (Maxiprep)

To isolate larger amounts of plasmid-DNA, the maxiprep method was performed. Two plungers with 100 ml of LB+ampicillin medium were inoculated with a colony or with 100 μl of a frozen culture, which carries the plasmid that is to be isolated, and it was incubated overnight at 37° C. and 120 rpm. The next day the culture (200 ml) was moved into a GSA beaker and centrifuged for 10 minutes at 4000 rpm (2600×g). The cell pellet was taken up in 6 ml of TE-buffer. To digest the cell wall, 1.2 ml of lysozyme solution (20 mg/ml of TE-buffer) was added, and it was incubated for 10 minutes at room temperature. Then, the lysis of the cells was carried out with 12 ml of 0.2N NaOH, 1% SDS solution and for another 5 minutes of incubation at room temperature. The proteins were precipitated by the addition of 9 ml of cooled 3 M sodium acetate solution (pH 4.8) and a 15-minute incubation on ice. After centrifuging (GSA: 13,000 rpm (27,500×g), 20 minutes, 4° C.), the supernatant, which contained the DNA, was moved into a new GSA beaker, and the DNA was precipitated with 15 ml of ice-cold isopropanol and an incubation of 30 minutes at −20° C. The DNA pellet was washed in 5 ml of ice-cooled ethanol and dried in air (about 30-60 minutes). Then, it was taken up in 1 ml of H₂O. An examination of the plasmid by restriction analysis took place. The concentration was determined by depositing dilutions on a minigel. To reduce the salt content, a 30-60 minute microdialysis was carried out (pore size 0.025 μm).

9. Yeast Transformation

For the yeast transformation, a pre-culture of the strain Saccharomyces cerevisiae (S. cerevisiae) AH22 was prepared. A plunger with 20 ml of YE-medium was inoculated with 100 μl of the frozen culture and incubated overnight at 28° C. and 120 rpm. The main cultivation was carried out under identical conditions in a plunger with 100 ml of YE-medium, which was inoculated with 10 μl, 20 μl or 50 μl of the pre-culture.

9.1 Producing Competent Cells

The next day, the plungers were counted out using a Thoma chamber, and the procedure was continued with the plunger, which held 3-5×10⁷ cells/ml. The cells were harvested by centrifuging (GSA: 5000 rpm (4000×g), 10 minutes). The cell pellet was taken up in 10 ml of TE-buffer and divided into two tabletop centrifuging tubes (5 ml each). The cells were centrifuged off for 3 minutes at 6000 rpm and washed twice more with 5 ml of TE-buffer each. Then, the cell pellet was taken up in 330 μl of lithium acetate buffer per 10⁹ cells, moved into a sterile 50 ml Erlenmeyer flask and shaken for one hour at 28° C. As a result, the cells were competent for transformation.

9.2 Transformation

For each transformation preparation, 15 μl of herring sperm DNA (10 mg/ml), 10 μl of DNA that is to be transformed (about 0.5 μg) and 330 μl of component cells were pipetted into a tabletop centrifuging tube and incubated for 30 minutes at 28° C. (without shaking!). Then, 700 μl of 50% PEG 6000 was added, and it was incubated for one additional hour at 28° C., without shaking. A thermal shock of 5 minutes at 42° C. followed.

100 μl of the suspension was flattened out on the selection medium (YNB, Difco) to select leukine prototrophy. In the case of the selection on G418 resistance, a regeneration of the cells is carried out after the thermal shock (see under 9.3 Regeneration Phase).

9.3 Regeneration Phase

Since the selection marker is resistance to G418, the cells needed time for the expression of the resistance-gene. The transformation preparations were mixed with 4 ml of YE-medium and incubated overnight at 28° C. in the shaker (120 rpm). The next day, the cells were centrifuged off (6,000 rpm, 3 minutes), taken up in 1 ml of YE-medium, and 100 μl or 200 μl was flattened out on YE+G418 plates. The plates were incubated for several days at 28° C.

10. Reaction Conditions for the PCR

The reaction conditions for the polymerase chain reaction must be optimized for the individual case and are not necessarily valid for any batch. Thus, i.a., the amount of DNA used, the salt concentrations and the melting temperature can be varied. For our formulation of the problem, it has proven advantageous to combine the following substances in an Eppendorf cap, which was suitable for use in a thermocycler: 5 μl of super buffer, 8 μl of dNTP's (0.625 μM each), 5′-primer, 3′-primer and 0.2 μg of matrix DNA, dissolved in enough water to yield a total volume of 50 μl for the PCR preparation, were added to 2 μl (−0.1 U) of Super Taq polymerase. The batch was briefly centrifuged off and covered with a drop of oil. Between 37 and 40 cycles were selected for amplification.

The embodiments below describe the production of the plasmids and yeast strains according to the invention as well as their use, without, however, limiting the invention to these examples.

### EXAMPLE 1

### Expression of tHMG in S. cerevisiae AH22

The DNA sequence for tHMG (Basson et al., (1988)) was amplified by PCR from genomic DNA of Saccharomyces cerevisiae S288C. (Mortimer and Johnston, (1986)) with use of standard methods. The primers that are used in this case are the DNA oligomer tHMG-5′ and tHMG-3′ (see Seq ID Nos. 1 and 2). The DNA-fragment that was obtained was introduced in cloning vector pUC19 (Yanisch-Perron et al., (1985)) after a Klenow treatment, and yielded vector pUC19-tHMG. After plasmid isolation and restriction of pUC 19-tHMG with endonucleases EcoRl and BamHl, the obtained fragment was introduced into yeast expression vector pPT2b (Lang and Looman, (1995)), which also was treated with EcoRl and BamHl. The plasmid pPT2b-tHMG that was produced contains the ADH1-promoter (Bennetzen and Hall, (1982)) and the TRP1-terminator (Tschumper and Carbon, (1980)), between which the tHMG-DNA fragment is found. A DNA section was isolated from vector pPT2b-tHMG via endonucleases EcoRV and Nrul, and said DNA section contains the so-called average ADH1-promoter, the tHMG and the TRP1-terminator. This DNA section was introduced into yeast vector YEp13 (Fischhoff et al., (1984)), which was treated with endonuclease Sphl and a DNA polymerase. The vector that is thus produced, the YEpH2 (FIG. 1), was treated with the endonucleases EcoRV and Nrul. A DNA-fragment with the following areas was thus produced: a transcription-activating area from the tetracycline resistance gene (Sidhu and Bollon, (1990)), the average ADH1-promoter, the tHMG and the TRP1-terminator (expression cassette). This DNA-fragment was introduced into vector YDpU (Berben et al., (1991)), which was treated with Stul. Vector YDpUH2/12 that was thus produced was treated with endonuclease Smal and ligated with a DNA-sequence that codes for a kanamycin resistance (Webster and Dickson, (1983)). The construct that is produced (YDpUHK3, FIG. 2) was treated with EcoRV. The yeast strain Saccharomyces cerevisiae AH22 was transformed with this construct. The transformation of the yeast with a linearized vector, as it is in this example, results in a chromosomal integration of the total vector at the URA3 gene locus. To eliminate the areas from the integrated vector that are not part of the expression cassette (E. coli origin, E. coli-ampicillin resistance gene, TEF-promoter and kanamycin resistance gene), transformed yeasts were subjected to a selection pressure by FOA selection (Boeke et al., (1987)) that promotes uracil-auxotrophic yeasts. The uracil-auxotrophic strain that is described in the selection bears the name AH22/tH3ura8 and has the tHMG1-expression cassette as chromosomal integration in the URA3-gene.

The yeast strain AH22/tH3ura8 and the starting strain AH22 were cultivated for 48 hours in YE at 28° C. and 160 rpm in a flow spoiler plunger.

Cultivation conditions: Pre-culture WMVIII was prepared as follows: 20 ml of WMVIII+histidine (20 μg/ml)+uracil (20 μg/ml) were inoculated with 100 μl of frozen culture and incubated for 2 days at 28° C. and 120 rpm (reciprocal motion). From the 20 ml of pre-culture, 100 ml of WMVIII+histidine (20 μg/ml)+uracil (20 μg/ml) were inoculated. For the main culture, 50 ml of YE (in a 250 ml flow spoiler plunger) with 1×10⁹ cells was inoculated. The plungers were incubated at 160 rpm in a round shaker at 28° C. for 48 hours. HMG-CoA-reductase activities were determined (according to Qureshi et al., (1981)), and produced the following values (see Table 1).

TABLE 1

| | Specific HMG-CoA-reductase |
|---|---|
| | activity* (U/mg of protein) |
| AH22 | 3.99 |
| AH22/tH3ura8 | 11.12 |

*A unit is defined as the reaction of 1 nmol of NADPH per minute in a milliliter reaction mixture. The measurement was carried out with total protein isolates.

The sterols were extracted (Parks et al., (1985)) and analyzed using gas chromatography. The following values were produced (see Table 2).

TABLE 2

| | Squalene (% w/w) | Ergosterol (% w/w) |
|---|---|---|
| AH22 | 0.01794 | 1.639 |
| AH22/tH3ura8 | 0.8361 | 1.7024 |

The values, in percent, relate to the dry weight of the yeast.

### EXAMPLE 2

### Expression of SAT1 in S. cerevisiae AH22

The sequence for the acyl-CoA: sterol transferase (SAT1; Yang et al., (1996)) was obtained by, as described above, PCR from genomic DNA of Saccharomyces cerevisiae S288C. The primers used in this case are the DNA-oligomers SAT1-5′ and SAT1-3′ (see Seq ID Nos. 3 and 4). The DNA-fragment that was obtained was cloned in cloning vector pGEM-T (Mezei and Storts, (1994)), which resulted in vector pGEM-SAT1. By treatment of pGEM-SAT1 with EcoRl, a fragment was obtained that was cloned in yeast expression vector pADH1001, which also was treated with EcoRl. Vector pADH-SAT1 that was thus produced was treated with the endonuclease Nrul, and it was ligated with a fragment of YEp13, which contains the LEU2-gene.

Yeast expression vector pADL-SAT1 (FIG. 3), which was introduced into yeast strain AH22, was thus produced. The thus obtained strain AH22/pADL-SAT1 was incubated for 7 days in WMVIII (Lang and Looman (1995)) in a minimal medium. Cultivation conditions: (For pre-culture, see above) Main culture: 50 ml of WMVIII+histidine (20 μg/ml)+uracil (20 μg/ml) of cultures (in a 250 ml flow spoiler plunger) were inoculated with 1×10⁹ cells: the plungers were incubated at 160 rpm on a round shaker at 28° C. for 7 days. The sterols formed were analyzed via gas chromatography (see Table 3).

TABLE 3

| | Squalene (% w/w) | Ergosterol (% w/w) |
|---|---|---|
| AH22 | n.d. | 1.254 |
| AH22/pADL-SAT1 | n.d. | 1.831 |

The values, in percent, relate to the dry weight of the yeast.
n.d.: indeterminate

### EXAMPLE 3

### Combined Expression of the Shortened 3-Hydroxy-3-methylglutaryl-CoA-Reductase (tHMG) and the Acyl-CoA: Sterol-acyl Transferase (SAT1)

### EXAMPLE 3.1

Yeast strain AH22/tH3ura 8 was transformed with the SAT1 expression vector pADL-SAT1, and yielded AH22/tH3ura8/pADL-SAT1. This combined strain was cultivated for 7 days in WMVIII. The sterols were extracted (see above) and analyzed via gas chromatography. The following values were produced (see Table 4).

TABLE 4

| | Squalene (% w/w) | Ergosterol (% w/w) |
|---|---|---|
| AH22/tH3ura8 | 1.602 | 3.798 |
| AH22/tH3ura8/pADL- | 1.049 | 5.540 |
| SAT1 | | |

The values, in percent, relate to the dry weight of the yeast.

### EXAMPLE 3.2

Yeast cultures were cultivated for 7 days in WMVIII, but different amounts of uracil were added to the cultures. Concentrations of 10, 20, 40 and 100 μg/ml of uracil were set in the medium. The ergosterol and the squalene amounts are at most in a supplementation of 20 μg/ml of uracil. The results are shown in FIG. 4.

It is shown that the ergosterol and squalene yield in strain AH22tH3ura8/pADL-SAT1 depends greatly on the amount of uracil that is added to cultivation medium WMVIII.

### EXAMPLE 3.3

Yeast cultures were cultivated for 7 days in WMVIII. Then, the totality of the sterols was determined as described above. The free sterols are determined by GC from yeasts that are encapsulated with glass pearls and are extracted with n-hexane.

The results are shown in Table 5.

The results show that the enzyme sterol-acyl transferase (Sat1) esterifies with higher effectiveness in particular sterols that are lacking the 4,4-dimethyl group. Thus, a technical application for the separation of 4, -4-dimethylsterols from the corresponding demethylated forms is also suitable.

Table 5

Proportion by percentage of free sterols. Each sterol was determined as a free sterol (without solution) and was related to the total amount of this sterol. The absolute total sterol contents as area/g dry substance are indicated in parentheses. Lanosterol and 4,4-dimethylzymosterol are sterols with a 4,4-dimethyl group.

| | % of free sterols | |
|---|---|---|
| | Control | AH22tH3ura8/pADL-SAT1 |
| Lanosterol | 54 (0.99) | 59 (2.90) |
| 4,4-dimethylzymosterol | 58 (0.77) | 84 (2.37) |
| 4-methylzymosterol | 7 (2.43) | 10 (7.62) |
| zymosterol | 10 (1.67 | 11 (5.85) |
| ergost-7-enol | 24 (4.55) | 12 (9.00) |
| ergosta-5,7-dienol | | |

### DESCRIPTION OF THE FIGURES

FIG. 1 shows plasmid YEpH2 with the corresponding interfaces.

FIG. 2 shows the plasmid YDpUHK3 with the corresponding interfaces.

FIG. 3 shows the plasmid pADL-SAT1 with the corresponding interfaces.

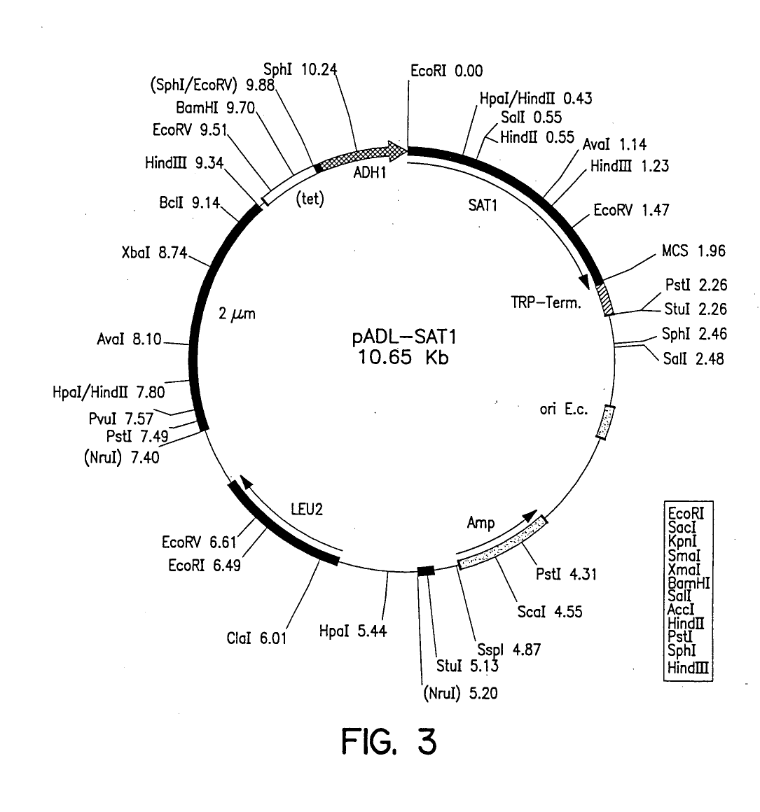

FIG. 4 shows the growth behavior and ergosterol and squalene contents with different uracil supplementation. In the figure, OD=optical density, Kultivierungszeit=cultivation time, Hefe-Trockengewicht=yeast dry weight, Uracilsupplementation=uracil supplementation.

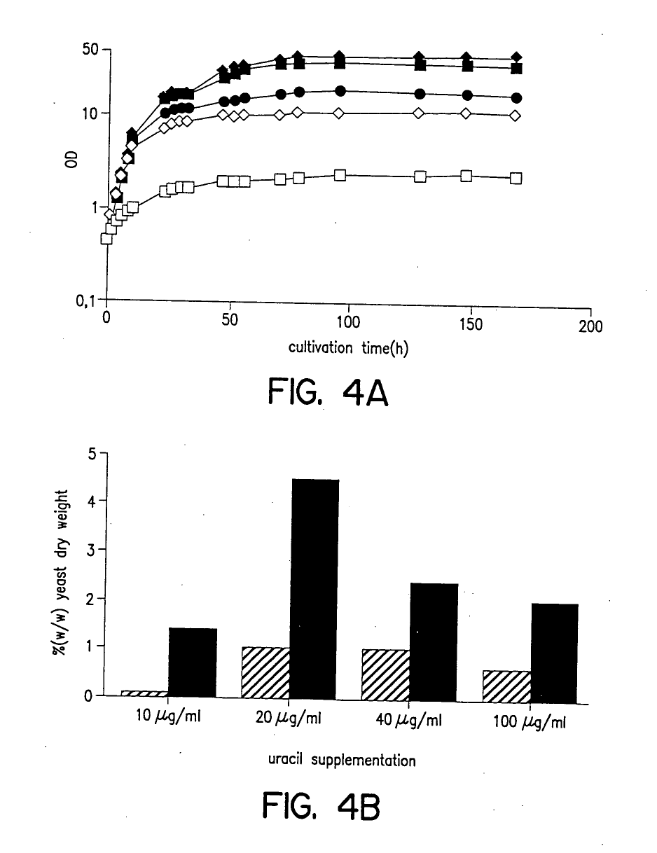

### Bibliographic References

Basson, M. E.; Thorsness, M.; Finer-Moore, J.; Stroud, R. M.; Rine, J. (1988) Structural and Functional Conservation between Yeast and Human 3-Hydroxy-3-methylglutaryl Coenzyme A Reductases, The Rate-limiting Enzyme of Sterol Biosynthesis. Mol. Cell. Biol. 8: 3793-3808.

Bennetzen, J. L.; Hall, B. D. (1982) The Primary Structure of the Saccharomyces cerevisiae Gene for Alcohol Dehydrogenase. J. Biol. Chem. 257: 3018-3025.

Berben, G.; Dumont, J.; Gilliquet, V.; Bolle, P. A.; Hilger, F. (1991) The YDp Plasmids: A Uniform Set of Vectors Bearing Versatile Gene Disruption Cassettes for Saccharomyces cerevisiae. Yeast 7: 475-477.

Boeke, J. D.; Trueheart, J.; Natsoulis, G.; Fink, G. (1987) 5-Fluorootic Acid as a Selective Agent in Yeast Molecular Genetics. Methods in Enzymology 154: 164-175.

Fegueur, M.; Richard, L.; Charles, A. D.; Karst, F. (1991) Isolation and Primary Structure of the ERG9 Gene of Saccharomyces cerevisiae Encoding Squalene Synthetase. Current Genetics 20: 365-372.

Fischhoff, D. A.; Waterston, R. H.; Olson, M. V. (1984) The Yeast Cloning Vector YEp13 Contains a tRNALeu3 Gene That Can Mutate to an Amber Suppressor. Gene 27: 239-251.

Jandrositz, A.; Turnowsky, F.; Högenauer, G. (1991) The Gene Encoding Squalene Epoxidase from Saccharomyces cerevisiae: Cloning and Characterization. Gene 107: 155-160.

Mezei, L. M.; Storts, D. R. (1994) in: PCR Technology: Current Innovations, Griffin, H. G. and Griffin, A. M., eds. CRC Press, Boca Raton, 21.

Mortimer, R. K.; Johnston, J. R. (1986) Genealogy of Principal Strains of the Yeast Genetic Stock Center. Genetics 113: 35-43.

Lang, C.; Looman, A. C. (1995) Efficient Expression and Secretion of Aspergillus niger RH5344 Polygalacturonase in Saccharomyces cerevisiae. Appl. Microbiol. Biotechnol. 44: 147-156.

Parks, L. W.; Bottema, C. D. K., Rodriguez, R. J.; Lewis, T. A. (1985) Yeast Sterols: Yeast Mutants as Tools for the Study of Sterol Metabolism. Meth. Enzymol. 111, 333-346.

Qureshi, N.; Nimmannit, S.; Porter, J. W. (1981) 3-Hydroxy-3-methylglutaryl-CoA Reductase from Yeast. Meth. Enzymol. 71: 455-461.

Ruohonen, L.; Aalto, M. K.; Keranen, S. (1995) Modifications to the ADH1-Promoter of Saccharomyces cerevisiae for Efficient Production of Heterologous Proteins. Journal of Biotechnology 39: 193-203.

Siduh, R. S.; Bollon, A. P. (1990) Bacterial Plasmid pBR322 Sequences Serve as Upstream Activating Sequences in Saccharomyces cerevisiae. Yeast 6: 221-229.

Tschumper, G.; Carbon, J. (1980) Sequence of a Yeast DNA Fragment Containing a Chromosomal Replicator and the TRP1 Gene. Gene 10: 157-166.

Webster, T. D.; Dickson, R. C. (1983) Direct Selection of Saccharomyces cerevisiae Resistant to the Antibiotic G418 Following Transformation with a DNA Vector Carrying the Kanamycin-Resistance Gene of Tn903. Gene 26: 243-252.

Yang, H.; Bard, M.; Bruner, D. A.; Gleeson, A.; Deckelbaum, R. J.; Aljinovic, G.; Pohl, T. M.; Rothstein, R.; Sturley, S. L. (1996) Sterol Esterification in Yeast: A Two-Gene Process. Science 272: 1353-1356.

Yanisch-Perron, C.; Vieira, J.; Messing, J. Gene 33 (1985) 103-119.

Yu, C.; Rothblatt, J. A. Cloning and Characterization of the Saccharomyces cerevisiae Acyl-CoA: Sterol-Acyl Transferase (1996). The Journal of Biological Chemistry, 271: 24157-24163.

1. Process for the production of ergosterol and its intermediate products, characterized in that
a) first a plasmid is designed, into which several suitable genes of the ergosterol metabolic process are inserted in altered form, or
b) first plasmids are designed, into which in each case one of the genes of the ergosterol metabolic process is inserted in altered from,
c) microorganisms are transformed with the thus produced plasmids, whereby the microorganisms are transformed with a plasmid under a) or they are transformed simultaneously or in succession with several plasmids under b),
d) fermentation into ergosterol is performed with the thus produced microorganisms,
e) after fermentation has ended, the ergosterol and its intermediate products are extracted from the cells and analyzed, and finally,
f) the thus obtained ergosterol and its intermediate products are purified using column chromatography and isolated.

2. Process according to claim 1, wherein
a-i) first a plasmid is designed, into which the following genes are inserted:
i) the gene of HMG-Co-A-reductase (t-HMG),
ii) the gene of squalene synthetase (ERG9),
iii) the gene of Acyl-CoA: sterol-acyl transferase (SAT1), and
iv) the gene of squalene epoxidase (ERG1), or
a-ii) first a plasmid is designed, into which the following genes are inserted:
i) the gene of HMG-Co-A-reductase (t-HMG), and
ii) the gene of squalene synthetase (ERG9), or
a-iii) first a plasmid is designed, into which the following genes are inserted:
i) the gene of. HMG-Co-A-reductase (t-HMG), and
iii) the gene of acyl-CoA: sterol-acyl transferase (SAT1), or
a-iv) first a plasmid is designed, into which the following genes are inserted:
i) the gene of the HMG-Co-A-reductase (t-HMG), and
iv) the gene of squalene epoxidase (ERG1), or
a-v) first a plasmid is designed, into which the following genes are inserted:
ii) the gene of squalene synthetase (ERG9), and
iii) the gene of acyl-CoA: sterol-acyl transferase (SAT1) or
a-vi) first a plasmid is designed, into which the following genes are inserted:
ii) the gene of squalene synthetase (ERG9), and
iv) the gene of squalene epoxidase (ERG1), or
a-vii) first a plasmid is designed, into which the following genes are inserted:
iii) the gene of acyl-CoA: sterol-acyl transferase (SAT1), and
iv) the gene of squalene epoxidase (ERG1), or
b) first plasmids are designed, into which in each case one of the genes that is mentioned under a-i) is inserted, and
c) microorganisms are transformed with the thus produced plasmids, whereby the microorganisms are transformed with a plasmid under a-i) to a-vii), or they are transformed simultaneously or in succession with several plasmids under b),
d) fermentation into ergosterol is performed with the thus produced microorganisms,
e) after fermentation has ended, the ergosterol and its intermediate products are extracted from the cells and analyzed, and finally
f) the thus obtained ergosterol and its intermediate products are purified using column chromatography and isolated.

3. Process according to claim 2, wherein in addition the gene of squalene epoxidase (ERG1) is inserted into the plasmid under a-ii), a-iii) and a-v), and in addition the gene of the acyl-CoA: sterol-acyl transferase is inserted into plasmid a-ii).

4. Process for the production of ergosterol and its intermediate products, wherein the genes that are mentioned in claim 1 under a), those in claim 2 under a-i) to a-vii) and those in claim 3 under a-ii), a-iii) and a-v) in each case with the plasmids are first introduced independently of one another into microorganisms of the same species, and fermentation into ergosterol is performed with them together and the ergosterol that is thus obtained is extracted from the cells, analyzed and purified using column chromatography and isolated.

5. Process according to claims 1 to 4, wherein the intermediate products are squalene, farnesol, geraniol, lanosterol, zymosterol, 4,4-dimethylzymosterol, 4-methylzymosterol, ergost-7-enol and ergosta-5,7-dienol.

6. Process according to claims 1 to 4, wherein the intermediate products are sterols with 5,7-diene structure.

7. Process according to claims 1 to 4, wherein the plasmids are plasmids YEpH2, YDpUHK3 and pADL-SAT1.

8. Process according to claims 1 to 4, wherein the microorganisms are yeasts.

9. Process according to claim 8, wherein it is the species S. cerevisiae.

10. Process according to claim 9, wherein it is the strain S. cerevisiae AH22.

11. Yeast strain S. cerevisiae AH22 that contains one or more of the genes that are mentioned under a-i) in the process.

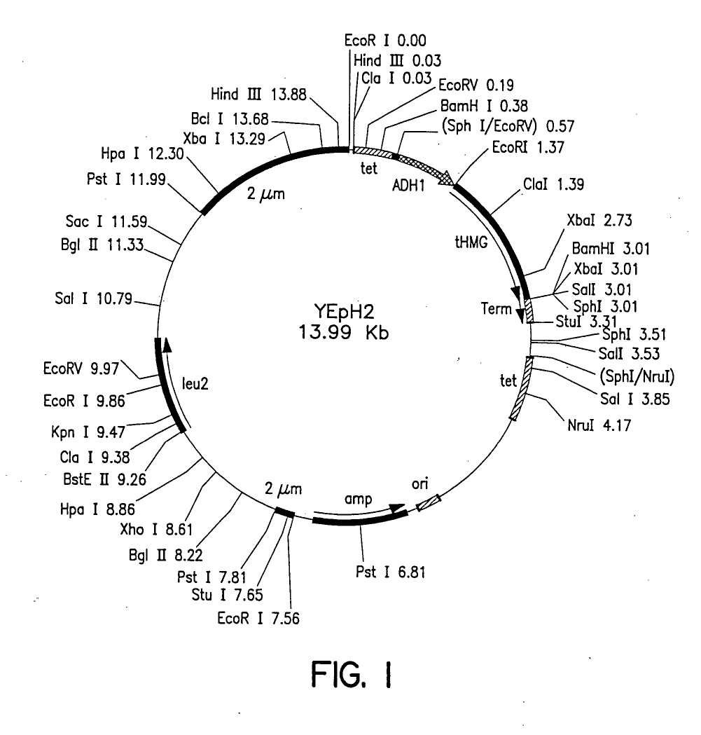

12. Plasmid YEpH2 that consists of the average ADH-promoter, t-HMG (altered variant of HMG-1) and the TRP-terminator (FIG. 1).

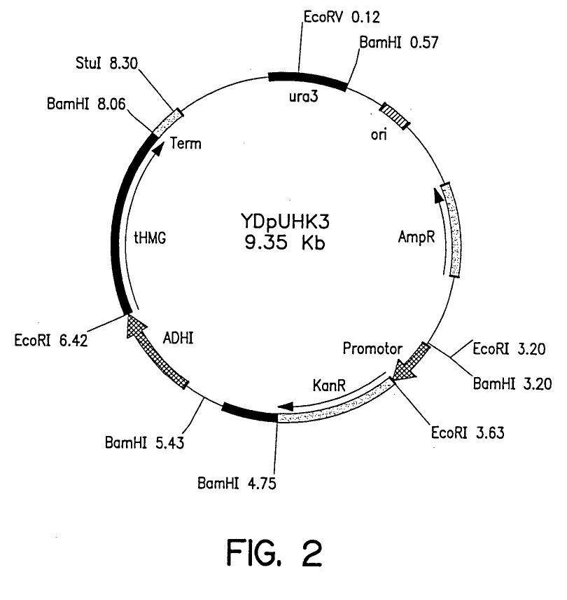

13. Plasmid YDpUHK3 that consists of the average ADH-promoter, t-HMG (altered variant of the HMG-1) and the TRP-terminator, the gene for the kanamycin resistance and the ura3 gene (FIG. 2).

14. Plasmid pADL-SAT1 that consists of the SAT1 gene and the LEU2 gene of YEp13.

15. Use of the plasmids according to claims 12 to 14 for the production of ergosterol.

16. Use of the plasmids according to claims 12 to 14 for the production of ergosterol intermediate products squalene, farnesol, geraniol, lanosterol, zymosterol, 4,4-dimethylzymosterol, 4-methylzymosterol, ergost-7-enol and ergosta-5,7-dienol.

17. Use of the plasmids according to claims 12 to 14 for the production of sterols with 5,7-diene structure.

18. Expression cassette that comprises the average ADH-promoter, the t-HMG gene, the TRP-terminator and the SAT1 gene with the average ADH-promoter and the TRP-terminator.

19. Expression cassette that comprises the average ADH-promoter, the t-HMG gene, the TRP-terminator, the SAT1 gene with the average ADH-promoter and the TRP-terminator, and the ERG9-gene with the average ADH-promoter and the TRP-terminator.

20. Combination of expression cassettes, whereby the combination consists of
a) a first expression cassette, on which the ADH-promoter, the t-HMG-gene, and the TRP-terminator are located,
b) a second expression cassette, on which the ADH-promoter, the SAT1-gene and the TRP-terminator are located, and
c) a third expression cassette, on which the ADP-promoter, and the ERG9-gene with the TRP-terminator are located.

21. Use of the expression cassettes according to claims 18 to 20, for the transformation of microorganisms, which are used in the fermentation into ergosterol.

22. Use according to claim 21, wherein the microorganism is yeast.

23. Microorganisms that contain expression cassettes according to claims 18 to 20.

24. Microorganism according to claim 23, wherein it is yeast.

25. Use of the microorganism according to claims 23 and 24 in the fermentation into ergosterol.

26. Use of the microorganism according to claims 23 and 24, in the fermentation into ergosterol intermediate products.


SEQUENCE LISTING (4 sequences)

SEQ ID NO: 1 - DNA, 25 bp, Artificial Sequence
  Description of Artificial Sequence Primer
actatggaccaattggtgaaaactg

SEQ ID NO: 2 - DNA, 23 bp, Artificial Sequence
  Description of Artificial Sequence Primer
agtcacatggtgctgttgtgctt

SEQ ID NO: 3 - DNA, 25 bp, Artificial Sequence
  Description of Artificial Sequence Primer
gaattcaaccatggacaagaagaag

SEQ ID NO: 4 - DNA, 24 bp, Artificial Sequence
  Description of Artificial Sequence Primer
agaattccacagaacagttgcagg